(19) United States
(12) Patent Application Publication
(10) Pub. No.: US 2026/0062019 A1
(43) Pub. Date: Mar. 5, 2026

(54) SYSTEMS AND METHODS FOR VEHICLE BIOMETRICS PROCESSING AND VEHICLE CONTROL

(71) Applicant: Toyota Motor Engineering & Manufacturing North America, Inc., Plano, TX (US)

(72) Inventors: Frederico Marcolino Quintao Severgnini, Ann Arbor, MI (US); Kleanthis Avramidis, Los Angeles, CA (US); Parveen Singh, Auburn Hills, MI (US); Ercan M. Dede, Ann Arbor, MI (US); Paul D. Schmalenberg, Ann Arbor, MI (US)

(73) Assignee: Toyota Motor Engineering & Manufacturing North America, Inc., Plano, TX (US)

(21) Appl. No.: 18/817,888

(22) Filed: Aug. 28, 2024

(51) Int. Cl. B60W 50/08; A61B 5/00; A61B 5/16; G06N 20/00

(52) U.S. Cl. CPC B60W 50/085; A61B 5/165; A61B 5/6893; A61B 5/7203; G06N 20/00

(57) ABSTRACT

A vehicle biometric system includes a processor and a non-transitory, processor-readable storage medium communicatively coupled to the processor, the non-transitory, processor-readable storage medium including one or more instructions stored thereon that, when executed, cause the processor to obtain benchmark biometric data from a first data source; obtain in-vehicle biometric data from a second data source; transmit the benchmark biometric data from the first data source and the in-vehicle biometric data from the second source to a neural network; update a model based on training data by processing the benchmark biometric data and the in-vehicle biometric data; transmit the updated model; ascertain at least one of an occupant condition and an emotional state of an occupant based on the updated model; and control one or more vehicle action events based on ascertaining at least one of the occupant condition and the emotional state of the occupant.

### FIELD

The present disclosure generally relates to devices for biometrics processing, and more particularly, to vehicle biometrics processing and vehicle control.

### BACKGROUND

Conventional techniques exist for monitoring data pertaining to an individual within a vehicle to estimate an individual condition. However, there are numerous challenges in obtaining and processing this data in an efficient manner. These and other deficiencies exist.

### BRIEF DESCRIPTION OF THE DRAWINGS

In one aspect, a vehicle biometric system may include a processor, and a non-transitory, processor-readable storage medium communicatively coupled to the processor, the non-transitory, processor-readable storage medium comprising one or more instructions stored thereon that, when executed, cause the processor to: obtain benchmark biometric data from a first data source; obtain in-vehicle biometric data from a second data source; transmit the benchmark biometric data from the first data source and the in-vehicle biometric data from the second source to a neural network; update a model based on training data by processing the benchmark biometric data and the in-vehicle biometric data; transmit the updated model; ascertain at least one of an occupant condition and an emotional state of an occupant based on the updated model; and control one or more vehicle action events based on ascertaining at least one of the occupant condition and the emotional state of the occupant.

In another aspect, a method may include obtaining benchmark biometric data from a first data source. The method may include obtaining in-vehicle biometric data from a second data source. The method may include transmitting the benchmark biometric data from the first data source and the in-vehicle biometric data from the second source to a neural network. The method may include updating a model based on training data by processing the benchmark biometric data and the in-vehicle biometric data. The method may include transmitting the updated model. The method may include ascertaining at least one of an occupant condition and an emotional state of an occupant based on the updated model. The method may include controlling one or more vehicle action events based on ascertaining at least one of the occupant condition and the emotional state of the occupant.

In another aspect, a non-transitory, computer-readable medium including instructions that, when executed by at least one processor, cause the at least one processor to perform one or more operations including obtaining benchmark biometric data from a first data source; obtaining in-vehicle biometric data from a second data source; transmitting the benchmark biometric data from the first data source and the in-vehicle biometric data from the second source to a neural network; updating a model based on training data by processing the benchmark biometric data and the in-vehicle biometric data; transmitting the updated model; ascertaining at least one of an occupant condition and an emotional state of an occupant based on the updated model; and controlling one or more vehicle action events based on ascertaining at least one of the occupant condition and the emotional state of the occupant.

These and other features, and characteristics of the present technology, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the disclosure. As used in the specification and in the claims, the singular form of ‘a', 'an', and 'the’ include plural referents unless the context clearly dictates otherwise.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, wherein like structure is indicated with like reference numerals and in which:

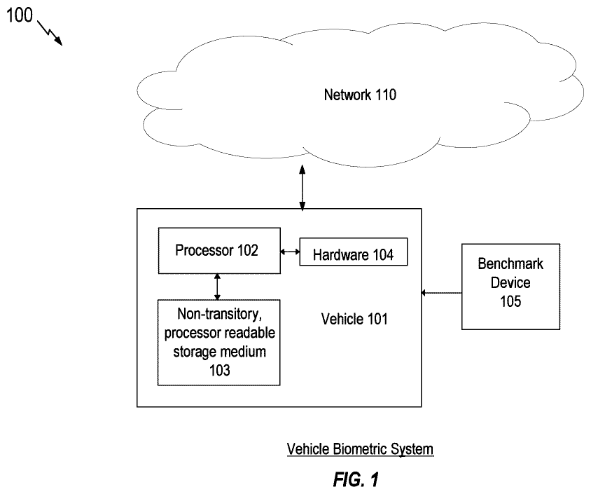

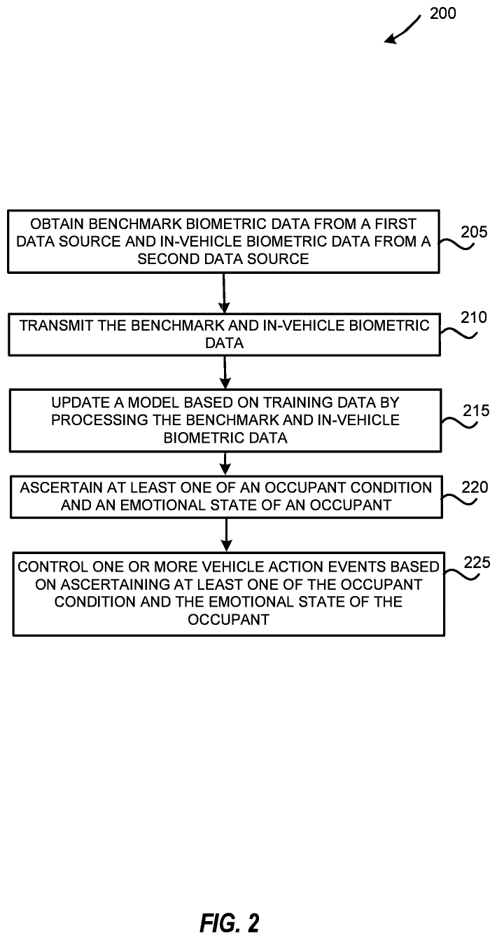

FIG. 1 depicts a schematic diagram of an example vehicle biometric system, according to one or more embodiments shown and described herein; and FIG. 2 depicts a flow diagram of an example method to be performed by a processor, according to one or more embodiments shown and described herein.

### DETAILED DESCRIPTION

The present disclosure relates to systems and methods for vehicle biometrics processing and vehicle control. Utilizing machine learning methods, the systems and methods disclosed herein may be configured to obtain and process respective biometric data from a truth data source (for example, a wearable device) and an in-vehicle data source (for example, in-vehicle hardware). By processing the biometric data from the truth data source and the in-vehicle data source within a neural network, the systems and methods disclosed herein improve the accuracy of an artificial intelligence system in determining a human condition and an emotional state of any number of occupants within a vehicle while also reducing the associated computationally processing intensive operations that would otherwise occur within a vehicle, thereby optimizing vehicle efficiency. The systems and methods disclosed herein are configured to generate and train the neural network to improve the accuracy of the determination of the human condition and the emotional state based on the respective biometric data from the truth and in-vehicle data sources, and in particular, by utilizing the truth data source as a calibration or a reference source with a greater signal-to-noise ratio for a given occupant. In certain embodiments, the systems and methods disclosed herein may no longer need a truth data source to be associated with an occupant to ascertain a biometric response of a given occupant, as the neural network can be trained to obtain and aggregate learning of sufficient biometric data to accurately predict or determine the biometric response of the given occupant, thereby reducing the amount of associated computationally processing intensive operations that would otherwise be needed from the truth data source. Further, the systems and methods disclosed herein are not limited to improving accuracy of only biometric response determination for an occupant within the vehicle, but can also be applied globally, for example to accurately predict or determine the biometric response for future or other occupants within the vehicle. Still further, the systems and methods disclosed herein are configured to take particular control and communication action with respect to the vehicle, in response to ascertaining the biometric response of the given occupant.

FIG. 1 depicts a schematic diagram of an example vehicle biometric system 100. As illustrated in FIG. 1, the vehicle biometric system 100 includes a vehicle 101, a benchmark device 105, and a network 110. The vehicle 101 may include a processor 102, a non-transitory processor readable storage medium 103, and hardware 104. Although FIG. 1 illustrates single instances of the constituent components of the vehicle biometric system 100, the vehicle biometric system 100 may include any number of constituent components.

In certain embodiments, the vehicle 101 may include an autonomous driving vehicle. In other embodiments, the vehicle 101 may include a vehicle that is not an autonomous driving vehicle. Without limitation, the vehicle may include a passenger vehicle, a non-passenger vehicle, a taxi, a bus, a scooter, a motorcycle, a truck, or any other type of vehicle. In certain embodiments, the vehicle 101 is not limited to a vehicle and that other types of objects may be instead included, such as a bed, a chair, a seat, a robot, medical device(s) and/or system(s), or the like. For example, the object, such as a bed, may be configured to monitor one or more vital parameters of a user, such as body temperature, respiration rate, blood pressure, pulse rate, blood oxygen, or any combination thereof, over a predetermined sleep schedule of the user.

The processor 102, such as a central processing unit (CPU), may be the central processing unit that is configured to perform calculations and logic operations to execute one or more programs. The processor 102, alone or in conjunction with the other components, may be an illustrative processing device, computing device, processor, or combinations thereof, including, for example, a multi-core processor, a microcontroller, a field-programmable gate array (FPGA), or an application-specific integrated circuit (ASIC). The processor 102 may include any processing component configured to receive and execute instructions (such as from the non-transitory processor readable storage medium 103). In some examples, the processor 102 may execute one or more applications, such as software applications, that enable, for example, network communications with one or more components of the system vehicle biometric system and transmit and/or receive data.

The non-transitory processor readable storage medium 103 may contain one or more data repositories for storing data that is received and/or generated. The non-transitory processor readable storage medium 103 may be any physical storage medium, including, but not limited to, a hard disk drive (HDD), memory (e.g., read-only memory (ROM), programmable read-only memory (PROM), random access memory (RAM), double data rate (DDR) RAM, flash memory, and/or the like), removable storage, a configuration file (e.g., text) and/or the like. While the non-transitory processor readable storage medium 103 is depicted as a local device, it should be understood that the non-transitory processor readable storage medium 103 may be a remote storage device, such as, for example, a server computing device, cloud-based storage device, or the like.

The hardware 104 may include one or more in-vehicle sensors. By way of example, the in-vehicle sensors may include any number and type of vehicle sensors that may be each configured to measure biometric data of a given occupant in a vehicle 101. Without limitation, the in-vehicle sensors may be configured to measure heart data, respiration data, eye information data, electrodermal activity data, or any combination thereof. Without limitation, the heart data may include heart rate data of the occupant. Without limitation, the eye information data may include eye movement data, blink rate data, or any combination thereof of the occupant. Without limitation, the electrodermal activity data may include electrical characteristics of skin of the occupant relative to a quantity of moisture and blood flow of the occupant. It is understood that other types of biometric data of the occupant may be measured by the in-vehicle sensors, and is not limited to these types of biometric data.

In certain embodiments, the hardware 104 may include one or more in-vehicle image acquisition devices. By way of example, the one or more in-vehicle image acquisition devices may include any number and type of vehicle cameras that may be each configured to obtain image acquisition data and/or video data corresponding to biometric data of the given occupant in the vehicle 101, including but not limited to the eye information data, the electrodermal activity data, the respiration data, the heart data, or any combination thereof of the occupant.

The benchmark device 105 may include one or more processing devices. By way of example, the processing device may be a network-enabled computer. As referred to herein, a network-enabled computer may include, but is not limited to a computer device, or communications device including, e.g., a server, a network appliance, a personal computer, a workstation, a phone, a wearable device including not limited to a wearable watch device, a handheld PC, a personal digital assistant, a thin client, a fat client, an Internet browser, or other device. The benchmark device 105 also may be a mobile device; for example, a mobile device may include an iPhone, iPod, iPad from Apple® or any other mobile device running Apple's iOS® operating system, any device running Microsoft's Windows® Mobile operating system, any device running Google's Android® operating system, and/or any other smartphone, tablet, or like wearable mobile device.

The benchmark device 105 can include a processor and a memory, similar or different than processor 102 and non-transitory processor readable storage medium 103, and it is understood that the processing circuitry may contain additional components, including processors, memories, error and parity/CRC checkers, data encoders, anticollision algorithms, controllers, command decoders, security primitives and tamperproofing hardware, as necessary to perform the functions described herein. The benchmark device 105 may further include a display and input devices. The display may be any type of device for presenting visual information such as a computer monitor, a flat panel display, and a mobile device screen, including liquid crystal displays, light-emitting diode displays, plasma panels, and cathode ray tube displays. The input devices may include any device for entering information into the user's device that is available and supported by the user's device, such as a touch-screen, keyboard, mouse, cursor-control device, touch-screen, microphone, digital camera, video recorder or camcorder. These devices may be used to enter information and interact with the software and other devices described herein.

The network 110 may be one or more of a wireless network, a wired network, or any combination of wireless network and wired network, and may be configured to operably communicate with any and all of the constituent components of the vehicle biometric system 100. For example, network 110 may include one or more of a fiber optics network, a passive optical network, a cable network, an Internet network, a satellite network, a wireless local area network (LAN), a Global System for Mobile Communication, a Personal Communication Service, a Personal Area Network, Wireless Application Protocol, Multimedia Messaging Service, Enhanced Messaging Service, Short Message Service, Time Division Multiplexing based systems, Code Division Multiple Access based systems, D-AMPS, Wi-Fi, Fixed Wireless Data, IEEE 802.11b, 802.15.1, 802.11n and 802.11g, Bluetooth, NFC, Radio Frequency Identification (RFID), Wi-Fi, and/or the like. In addition, the network 110 may include, without limitation, telephone lines, fiber optics, IEEE Ethernet 802.3, a wide area network, a wireless personal area network, a LAN, or a global network such as the Internet. In addition, the network 110 may support an Internet network, a wireless communication network, a cellular network, or the like, or any combination thereof. The network 110 may further include one network, or any number of the exemplary types of networks mentioned above, operating as a stand-alone network or in cooperation with each other. The network 110 may utilize one or more protocols of one or more network elements to which they are communicatively coupled. The network 110 may translate to or from other protocols to one or more protocols of network devices. Although the network 110 is depicted as a single network, it should be appreciated that in one or more aspects, the network 110 may include a plurality of interconnected networks, such as, for example, the Internet, a service provider's network, a cable television network, corporate networks, such as credit card association networks, and home networks. The network 110 may include a cloud-based network or a cloud network that can include a processor and a memory, similar or different than processor 102 and non-transitory processor readable storage medium 103.

The processor 102 may be configured to obtain benchmark biometric data from a first data source, such as the benchmark device 105. The processor 102 may be configured to obtain in-vehicle biometric data from a second data source, such as the vehicle 101 via the hardware 104. The benchmark biometric data from the benchmark device 105 and/or the in-vehicle biometric data from the vehicle 101 may be obtained in real-time. In certain embodiments, the processor 102 may be configured to continuously acquire the benchmark biometric data from the benchmark device 105, continuously acquire the in-vehicle biometric data from the vehicle 101. In certain embodiments, the processor 102 may be configured to periodically acquire the benchmark biometric data from the benchmark device 105, and periodically acquire the in-vehicle biometric data from the vehicle 101. By way of example and without limitation, the processor 102 may be configured to continuously or periodically obtain the benchmark biometric data and/or the in-vehicle biometric data every, for example, one second, three seconds, five seconds, ten seconds, or the like. It is understood that any predetermined time may be used for obtaining the benchmark biometric data and/or the in-vehicle biometric data, such as seconds, minutes, hours, or the like. It is understood that the benchmark biometric data and/or the in-vehicle biometric data may be obtained while the vehicle 101 is traveling, such as driving in a particular direction, and/or not traveling, such as being parked or stopped at an intersection.

In certain embodiments, it is understood that the manner of (for example, continuous or periodic) and predetermined time for obtaining the benchmark biometric data from the benchmark device 105 may be the same or different than the manner of and predetermined time for obtaining the in-vehicle biometric data from the vehicle 101. Still further, in certain embodiments, the contents of the biometric data obtained by the processor 102 may be the same or different from the benchmark device 105 and the vehicle 101. For example, the benchmark biometric data of the benchmark device 105 may include heart data and electrodermal activity data whereas the biometric data of the vehicle 101 may include eye information data. In another example, the benchmark biometric data of the benchmark device 105 and the biometric data of the vehicle 101 may both include respiration data.

The benchmark device 105 may include any number of sensors that are configured to measure biometric data, such as benchmark biometric data, of a given occupant in a vehicle 101. For example, the benchmark device 105 may include a wearable device, such as a smart watch or a smart band or a smart ring, worn by the occupant and configured to measure heart data, respiration data, eye information data, electrodermal activity data, or any combination thereof. Without limitation, the heart data may include heart rate data of the occupant. Without limitation, the eye information data may include eye movement data, blink rate data, or any combination thereof of the occupant. Without limitation, the electrodermal activity data may include electrical characteristics of skin of the occupant relative to a quantity of moisture and blood flow of the occupant.

It is understood that other types of biometric data of the occupant may be measured by the benchmark device 105, and is not limited to these types of biometric data. In certain embodiments, the benchmark device 105 may not need to be worn by the occupant within the vehicle 101 and can still be configured to measure the biometric data of the occupant within the vehicle 101. In certain embodiments, the benchmark device 105 may provide a more accurate measurement of biometric data of the occupant of the vehicle as compared to that which is obtained by the hardware 104 of the vehicle 101. For example, the benchmark device 105 may be configured to measure the biometric data of the occupant and derive a signal with a greater signal-to-noise ratio (SNR) than a signal of biometric data of the occupant obtained from the vehicle 101. In certain embodiments, the benchmark biometric data from the benchmark device 105 may have a better SNR than that of the in-vehicle biometric data from the vehicle 101 due to, for example, the benchmark device 105 being in closer proximity or more direct contact with the body of the occupant. Such measurements can also be taken into account in teaching a learning model how much noise from the in-vehicle biometric data of the vehicle 101 can be removed.

The processor 102 may be configured to transmit the benchmark biometric data from the benchmark device 105 and the in-vehicle biometric data from the vehicle 101 to an artificial intelligence model, such as a neural network, via the network 110. In certain embodiments, the processor 102 may be configured to pre-process the benchmark biometric data and the in-vehicle biometric data to filter out noise and thereby produce a reduced noise signal to the neural network. Without limitation, the noise removal from the signals of the benchmark biometric data and the in-vehicle biometric data may be performed by a filter, such as band pass filter. Further, the neural network may be trained, tested, fine-tuned, or any combination thereof, as further explained below.

Still further, the processor 102 may be configured to provide a portion of the benchmark biometric data and/or a portion of the in-vehicle biometric data to the neural network. Such portion of the respective biometric data may include a portion of raw data that is transmitted to the neural network. For example, in such a case, the raw data of ECG waveform may be selectively parsed by the processor 102 to include a portion thereof, such as a position of the heartbeats, instead of transmitting the complete ECG waveform to the neural network. In another example, the raw data may be unnecessarily complex and/or represent a large data file, in which case the processor 102 may be configured to compress the large data file and/or simplify the complex raw data prior to transmitting it to the neural network. In this manner, the processor 102 may be configured to increase operational processing efficiency by reducing the bandwidth needed to transmit data to and from the neural network.

The processor 102 may be configured to update a model associated with the neural network by processing the benchmark biometric data and the in-vehicle biometric data. In certain embodiments, the processor 102 may be configured to generate one or more prompts related to a state of the occupant. For example, the processor 102 may be configured to generate a prompt related to requesting an occupant to self-report their state. By way of example, the prompt may include: “How are you feeling today?” or “How are you feeling during this ride?” or “Please select which present state you are experiencing”. It is understood that these prompts are exemplary, and that open-ended or closed-ended prompts or any combination thereof may be transmitted. Further, the occupant may be provided an option of selecting and/or inputting the corresponding state regarding themselves and with reference to the one or more prompts, via user input as explained below. The processor 102 may be configured to transmit the one or more prompts related to the state of the occupant, as explained below.

The processor 102 may be configured to receive, in response to the one or more prompts, one or more responses via user input. Without limitation, the user input may include audio input, textual input, or any combination thereof. In certain embodiments, the occupant may receive the one or more prompts via their mobile device or benchmark device 105. In certain embodiments, the occupant may receive the one or more prompts via in-vehicle equipment of the vehicle 101, such as a physical or touch display panel of the vehicle 101 and/or an audio system of the vehicle 101. In this manner, the occupant may provide, via their mobile device or benchmark device 105 or via the display panel or the audio system of the vehicle 101, or any combination thereof, one or more responses to the one or more prompts. Still further, in certain embodiments, the occupant may provide verbal confirmation and/or gesture confirmation (such as via a nod, a thumbs up, or the like) regarding how they are feeling (as part of the user input) in response to the one or more prompts that are generated and transmitted.

Based on the one or more responses received via the user input, the processor 102 may be configured to update the model and transmit the updated model. For example, the processor 102 may be configured to update the model by aggregating user-feedback received from the occupant via the user input pertaining to their self-reported state, and analyzing it with respect to the benchmark biometric data from the benchmark device 105 and/or the in-vehicle biometric data from the vehicle 101. By continuing to receive and aggregate the received biometric data from the benchmark device 105, the vehicle 101, and/or the self-reported state of the occupant of the vehicle 101, the model is configured to more accurately determine how the biometric data is related to, for example, an anxiety attack and also prior to undertaking control of one or more vehicle action events associated with the vehicle 101. The processor 102 may be configured to transmit the updated model. For example, the vehicle 101 may be configured to receive the updated model from the neural network via the network 110.

The processor 102 may be configured to ascertain at least one of an occupant condition and an emotional state of the occupant based on the updated model. For example, the updated model may include the benchmark biometric data, the in-vehicle biometric data, the user feedback data, or any combination thereof. Without limitation, the occupant condition may include degrees of drowsiness, fatigue, sleepiness, lethargy, or any combination thereof. It is understood that the condition of the occupant may include other conditions and is not limited to only these occupant conditions. Without limitation, the emotional state of the occupant may include degrees of stressed, relaxed, excited, anxious, nervous, happy, sad, confused, uncertain, afraid, surprised, irritable, angry, impatient, bored, pleased, or any combination thereof. It is understood that the emotional state of the occupant may include other states of emotions and is not limited to only these emotional states. The ascertaining of the at least one of the occupant condition and the emotional state of the occupant of the vehicle 101 may be performed in real-time by the processor 102.

In certain embodiments, the processor 102 may be configured to ascertain a health emergency that impair a driver occupant prior to controlling one or more vehicle action events associated with the vehicle. Based on the benchmark biometric data from the benchmark device 105, the in-vehicle biometric data from the vehicle 101, and/or the user-feedback received from the occupant via the user input pertaining to their self-reported state, the processor 102 may be configured to ascertain a heart attack, a seizure, sleepiness, drowsiness (such as intoxication), stroke, or the like, of a given occupant. It is understood that the processor 102 may be configured to ascertain at least one of the occupant condition and the emotional state of a plurality of occupants within the vehicle 101, and not just for a single occupant within the vehicle 101. Still further, the model is configured to receive and aggregate the received biometric data from the benchmark device 105, the vehicle 101, and/or the self-reported state of the occupant of the vehicle 101, and then compare it against reference biometric data that are known to, or predicted to infer, a particular occupant condition and/or an emotional state of the occupant based on a likelihood of match between the reference biometric data and the received biometric data. By way of example, the reference biometric data may be stored in the neural network and/or retrieved by the neural network. For example, regarding the likelihood of match analysis, the neural network may be trained with new data, and weights may be accordingly updated. When the new data or measurements is sent to the neural network, the neural network may interpret this new data (as part of an input) and classify (as part of an output) an occupant state and/or condition. Moreover, a degree of certainty that the neural network has for each occupant state and/or condition may be retrieved.

In certain embodiments, the learning model may be sufficiently trained with the benchmark biometric data from the benchmark device 105, the in-vehicle biometric data from the vehicle 101, and/or the user-feedback received from the occupant via the user input pertaining to their self-reported state, such that it may no longer need to receive the benchmark biometric data from the benchmark device 105, or in other instances, receive the benchmark biometric data from the benchmark device 105 under less frequent times.

The processor 102 may be configured to control, in response to ascertaining at least one of the occupant condition and the emotional state of the occupant, one or more vehicle action events associated with the vehicle 101. By way of example and without limitation, the one or more vehicle action events associated with the vehicle 101 may include adjusting a speed of the vehicle 101, initiating communication with an entity via a generated alert, controlling the vehicle 101 to drive to a side of a road, initiating brakes of a vehicle 101, controlling the steering wheel of the vehicle 101, playing a sound in the vehicle 101 at a predetermined sound level, illuminating a light of the vehicle 101 at a predetermined brightness, vibrating a seat portion or a steering wheel or an arm rest of the vehicle 101, or any combination thereof. For example, the communication with the entity may be automatically initiated and may include contacting an authority, such as a health authority, in which they are kept informed and updated on a potential health emergency. In addition, the one or more vehicle action events associated with the vehicle 101 may include (for example, in the context of autonomous or sim-autonomous vehicles), dynamically modifying a route to reach a hospital or an emergency center or a parking lot and having the vehicle 101 autonomously or semi-autonomously navigate along the modified route, as well as dynamically suggesting alternative routes that are less stressful for the occupant(s) in response to determining that the occupant(s) is detected to exceed a predetermined threshold value of stress (or other condition or state) and having the vehicle 101 autonomously or semi-autonomously navigate along the suggested alternative route. The one or more vehicle action events associated with the vehicle 101 may further include automatically initiating communication with entities other than health authorities (such as police) for a threat due to an external force (for example, an occupant experiencing a particular condition and/or state in response to an adjacent tailgating driver of another vehicle 101) and/or specialists (such as for a health crisis).

For example, the processor 102 may be configured to reduce the speed of the vehicle 101 to a predetermined speed limit in response to ascertaining that the occupant is feeling drowsy. In another example, the processor 102 may be configured to initiate and establish communication with an emergency call system by generating and transmitting an alert to a processing device associated with the emergency call system in response to ascertaining that the occupant is feeling sleepy. In yet another example, the processor 102 may be configured to control the vehicle 101 (such as when the vehicle is an autonomous vehicle or a semi-autonomous vehicle) or instruct the vehicle 101 (such as when the vehicle is not an autonomous vehicle) to undertake an action of driving to a side of a road in response to ascertaining that the occupant is feeling sick. In such a case, the processor 102 may be configured to control the vehicle 101 or instruct the vehicle 101 to drive to the side of the road after determining that it is appropriate to do so.

FIG. 2 depicts a flow diagram of an example method 200 performed by a processor, such as the processor 102. FIG. 2 may reference and incorporate any of the above constituent components and corresponding disclosure explained above with respect to FIG. 1, such as the example vehicle biometric system 100.

At block 205, the processor 102 obtains benchmark biometric data from a first data source and in-vehicle biometric data from a second data source. For example, the processor 102 may be configured to obtain benchmark biometric data from a first data source, such as the benchmark device 105. The processor 102 may be configured to obtain in-vehicle biometric data from a second data source, such as the vehicle 101 via the hardware 104. The benchmark biometric data from the benchmark device 105 and/or the in-vehicle biometric data from the vehicle 101 may be obtained in real-time. In certain embodiments, the processor 102 may be configured to continuously acquire the benchmark biometric data from the benchmark device 105, continuously acquire the in-vehicle biometric data from the vehicle 101. In certain embodiments, the processor 102 may be configured to periodically acquire the benchmark biometric data from the benchmark device 105, and periodically acquire the in-vehicle biometric data from the vehicle 101. By way of example and without limitation, the processor 102 may be configured to continuously or periodically obtain the benchmark biometric data and/or the in-vehicle biometric data every, for example, one second, three seconds, five seconds, ten seconds, or the like. It is understood that any predetermined time may be used for obtaining the benchmark biometric data and/or the in-vehicle biometric data, such as seconds, minutes, hours, or the like. It is understood that the benchmark biometric data and/or the in-vehicle biometric data may be obtained while the vehicle 101 is traveling, such as driving in a particular direction, and/or not traveling, such as being parked or stopped at an intersection.

In certain embodiments, it is understood that the manner of (for example, continuous or periodic) and predetermined time for obtaining the benchmark biometric data from the benchmark device 105 may be the same or different than the manner of and predetermined time for obtaining the in-vehicle biometric data from the vehicle 101. Still further, in certain embodiments, the contents of the biometric data obtained by the processor 102 may be the same or different from the benchmark device 105 and the vehicle 101. For example, the benchmark biometric data of the benchmark device 105 may include heart data and electrodermal activity data whereas the biometric data of the vehicle 101 may include eye information data. In another example, the benchmark biometric data of the benchmark device 105 and the biometric data of the vehicle 101 may both include respiration data.

At block 210, the processor 102 transmits the benchmark biometric data and the in-vehicle biometric data. For example, the processor 102 may be configured to transmit the benchmark biometric data from the benchmark device 105 and the in-vehicle biometric data from the vehicle 101 to an artificial intelligence model, such as a neural network, via the network 110. In certain embodiments, the processor 102 may be configured to pre-process the benchmark biometric data and the in-vehicle biometric data to filter out noise and thereby produce a reduced noise signal to the neural network. Without limitation, the noise removal from the signals of the benchmark biometric data and the in-vehicle biometric data may be performed by a filter, such as band pass filter. As explained herein, the neural network may be trained, tested, fine-tuned, or any combination thereof.

Still further, the processor 102 may be configured to provide a portion of the benchmark biometric data and/or a portion of the in-vehicle biometric data to the neural network. Such portion of the respective biometric data may include a portion of raw data that is transmitted to the neural network. For example, in such a case, the raw data of ECG waveform may be selectively parsed by the processor 102 to include a portion thereof, such as a position of the heartbeats, instead of transmitting the complete ECG waveform to the neural network. In another example, the raw data may be unnecessarily complex and/or represent a large data file, in which case the processor 102 may be configured to compress the large data file and/or simplify the complex raw data prior to transmitting it to the neural network. In this manner, the processor 102 may be configured to increase operational processing efficiency by reducing the bandwidth needed to transmit data to and from the neural network.

At block 215, the processor 102 updates a model based on training data by processing the benchmark biometric data and the in-vehicle biometric data. The processor 102 may be configured to update a model associated with the neural network by processing the benchmark biometric data and the in-vehicle biometric data. In certain embodiments, the processor 102 may be configured to generate one or more prompts related to a state of the occupant. For example, the processor 102 may be configured to generate a prompt related to requesting an occupant to self-report their state. By way of example, the prompt may include: “How are you feeling today?” or “How are you feeling during this ride?” or “Please select which present state you are experiencing”. It is understood that these prompts are exemplary, and that open-ended or closed-ended prompts or any combination thereof may be transmitted. Further, the occupant may be provided an option of selecting and/or inputting the corresponding state regarding themselves and with reference to the one or more prompts, via user input as explained below.

The processor 102 may be configured to receive, in response to the one or more prompts, one or more responses via user input. Without limitation, the user input may include audio input, textual input, or any combination thereof. In certain embodiments, the occupant may receive the one or more prompts via their mobile device or benchmark device 105. In certain embodiments, the occupant may receive the one or more prompts via in-vehicle equipment of the vehicle 101, such as a physical or touch display panel of the vehicle 101 and/or an audio system of the vehicle 101. In this manner, the occupant may provide, via their mobile device or benchmark device 105 or via the display panel or the audio system of the vehicle 101, or any combination thereof, one or more responses to the one or more prompts. Still further, in certain embodiments, the occupant may provide verbal confirmation and/or gesture confirmation (such as via a nod, a thumbs up, or the like) regarding how they are feeling (as part of the user input) in response to the one or more prompts that are generated and transmitted.

Based on the one or more responses received via the user input, the processor 102 may be configured to update the model and transmit the updated model. For example, the processor 102 may be configured to update the model by aggregating user-feedback received from the occupant via the user input pertaining to their self-reported state, and analyzing it with respect to the benchmark biometric data from the benchmark device 105 and/or the in-vehicle biometric data from the vehicle 101. By continuing to receive and aggregate the received biometric data from the benchmark device 105, the vehicle 101, and/or the self-reported state of the occupant of the vehicle 101, the model is configured to more accurately determine how the biometric data is related to, for example, an anxiety attack and also prior to undertaking control of one or more vehicle action events associated with the vehicle 101. The processor 102 may be configured to transmit the updated model. For example, the vehicle 101 may be configured to receive the updated model from the neural network via the network 110.

At block 220, the processor 102 ascertains at least one of an occupant condition and an emotional state of an occupant of a vehicle 101. For example, the processor 102 may be configured to ascertain at least one of an occupant condition and an emotional state of the occupant based on the updated model. For example, the updated model may include the benchmark biometric data, the in-vehicle biometric data, the user feedback data, or any combination thereof. Without limitation, the occupant condition may include degrees of drowsiness, fatigue, sleepiness, lethargy, or any combination thereof. It is understood that the occupant condition of the occupant may include other conditions and is not limited to only these occupant conditions. Without limitation, the emotional state of the occupant may include degrees of stressed, relaxed, excited, anxious, nervous, happy, sad, confused, uncertain, afraid, surprised, irritable, angry, impatient, bored, pleased, or any combination thereof. It is understood that the emotional state of the occupant may include other states of emotions and is not limited to only these emotional states. The ascertaining of the at least one of the occupant condition and the emotional state of the occupant of the vehicle 101 may be performed in real-time by the processor 102.

In certain embodiments, the processor 102 may be configured to ascertain a health emergency that impair a driver occupant prior to controlling one or more vehicle action events associated with the vehicle. Based on the benchmark biometric data from the benchmark device 105, the in-vehicle biometric data from the vehicle 101, and/or the user-feedback received from the occupant via the user input pertaining to their self-reported state, the processor 102 may be configured to ascertain a heart attack, a seizure, sleepiness, drowsiness (including but not limited to intoxication), stroke, or the like, of a given occupant. It is understood that the processor 102 may be configured to ascertain at least one of the occupant condition and the emotional state of a plurality of occupants within the vehicle 101, and not just for a single occupant within the vehicle 101. Still further, the model is configured to receive and aggregate the received biometric data from the benchmark device 105, the vehicle 101, and/or the self-reported state of the occupant of the vehicle 101, and then compare it against reference biometric data that are known to, or predicted to infer, a particular occupant condition and/or an emotional state of the occupant based on a likelihood of match between the reference biometric data and the received biometric data. By way of example, the reference biometric data may be stored in the neural network and/or retrieved by the neural network. For example, regarding the likelihood of match analysis, the neural network may be trained with new data and weights may be accordingly updated. When the new data or measurements is sent to the neural network, the neural network may interpret this new data (as part of an input) and classify (as part of an output) an occupant state and/or condition. Moreover, a degree of certainty that the neural network has for each occupant state and/or condition may be retrieved.

At block 225, the processor 102 controls, in response to ascertaining at last one of the occupant condition and the emotional state of the occupant, one or more vehicle action events. For example, the processor 102 may be configured to control, in response to ascertaining at least one of the occupant condition and the emotional state of the occupant, one or more vehicle action events associated with the vehicle 101. By way of example and without limitation, the one or more vehicle action events associated with the vehicle 101 may include adjusting a speed of the vehicle 101, initiating communication with an entity via a generated alert, controlling the vehicle 101 to drive to a side of a road, initiating brakes of a vehicle 101, controlling the steering wheel of the vehicle 101, playing a sound in the vehicle 101 at a predetermined sound level, illuminating a light of the vehicle 101 at a predetermined brightness, vibrating a seat portion or a steering wheel or an arm rest of the vehicle 101, or any combination thereof. For example, the communication with the entity may be automatically initiated and may include contacting an authority, such as a health authority, in which they are kept informed and updated on a potential health emergency. In addition, the one or more vehicle action events associated with the vehicle 101 may include (for example, in the context of autonomous or semi-autonomous vehicles), dynamically modifying a route to reach a hospital or an emergency center or a parking lot and having the vehicle 101 autonomously or semi-autonomously navigate along the modified route, as well as dynamically suggesting alternative routes that are less stressful for the occupant(s) in response to determining that the occupant(s) is detected to exceed a predetermined threshold value of stress (or other condition or state) and having the vehicle 101 autonomously or semi-autonomously navigate along the suggested alternative route. The one or more vehicle action events associated with the vehicle 101 may further include automatically initiating communication with entities other than health authorities (such as police) for a threat due to an external force (for example, an occupant experiencing a particular condition and/or state in response to an adjacent tailgating driver of another vehicle 101) and/or specialists (such as for a health crisis).

For example, the processor 102 may be configured to reduce the speed of the vehicle 101 to a predetermined speed limit in response to ascertaining that the occupant is feeling drowsy. In another example, the processor 102 may be configured to initiate and establish communication with an emergency call system by generating and transmitting an alert to a processing device associated with the emergency call system in response to ascertaining that the occupant is feeling sleepy. In yet another example, the processor 102 may be configured to control the vehicle 101 (such as when the vehicle is an autonomous vehicle or a semi-autonomous vehicle) or instruct the vehicle 101 (such as when the vehicle is not an autonomous vehicle) to undertake an action of driving to a side of a road in response to ascertaining that the occupant is feeling sick. In such a case, the processor 102 may be configured to control the vehicle 101 or instruct the vehicle 101 to drive to the side of the road after determining that it is appropriate to do so.

The present disclosure relates to systems and methods for vehicle biometrics processing and vehicle control. Utilizing machine learning methods, the systems and methods disclosed herein may be configured to obtain and process respective biometric data from a truth data source (for example, a wearable device) and an in-vehicle data source (for example, in-vehicle hardware). By processing the biometric data from the truth data source and the in-vehicle data source within a neural network, the systems and methods disclosed herein improve the accuracy in determining a human condition and an emotional state of any number of occupants within a vehicle while also reducing the associated computationally processing intensive operations that would otherwise occur within a vehicle, thereby optimizing vehicle efficiency. The systems and methods disclosed herein are configured to generate and train the neural network to improve the accuracy of the determination of the human condition and the emotional state based on the respective biometric data from the truth and in-vehicle data sources, and in particular, by utilizing the truth data source as a calibration or a reference source with a greater signal-to-noise ratio for a given occupant. In certain embodiments, the systems and methods disclosed herein may no longer need a truth data source to be associated with an occupant to ascertain a biometric response of a given occupant, as the neural network can be trained to obtain and aggregate learning of sufficient biometric data to accurately predict or determine the biometric response of the given occupant, thereby reducing the amount of associated computationally processing intensive operations that would otherwise be needed from the truth data source. Further, the systems and methods disclosed herein are not limited to improving accuracy of only biometric response determination for an occupant within the vehicle, but can also be applied globally, for example to accurately predict or determine the biometric response for future or other occupants within the vehicle. Still further, the systems and methods disclosed herein are configured to take particular control and communication action with respect to the vehicle, in response to ascertaining the biometric response of the given occupant.

Further aspects of the disclosure are provided by the subject matter of the following clauses.

A vehicle biometric system, comprising: a processor; and a non-transitory, processor-readable storage medium communicatively coupled to the processor, the non-transitory, processor-readable storage medium comprising one or more instructions stored thereon that, when executed, cause the processor to: obtain benchmark biometric data from a first data source; obtain in-vehicle biometric data from a second data source; transmit the benchmark biometric data from the first data source and the in-vehicle biometric data from the second source to a neural network; update a model based on training data by processing the benchmark biometric data and the in-vehicle biometric data; transmit the updated model; ascertain at least one of an occupant condition and an emotional state of an occupant based on the updated model; and control one or more vehicle action events based on ascertaining at least one of the occupant condition and the emotional state of the occupant.

The vehicle biometric system of the previous clause, wherein the one or more instructions further cause the processor to pre-process the benchmark biometric data and the in-vehicle biometric data to filter noise.

The vehicle biometric system of any of the previous clauses, wherein the benchmark biometric data or the in-vehicle biometric data includes heart data, respiration data, eye information data, electrodermal activity data, or any combination thereof.

The vehicle biometric system of any of the previous clauses, wherein the one or more instructions further cause the processor to: generate one or more prompts related to a state of the occupant; transmit the one or more prompts related to the state of the occupant; and receive, in response to the one or more prompts, one or more responses via user input.

The vehicle biometric system of any of the previous clauses, wherein the user input includes audio input, textual input, gesture input, or any combination thereof.

The vehicle biometric system of any of the previous clauses, wherein the one or more instructions further cause the processor to update the model based on the one or more responses via the user input.

The vehicle biometric system of any of the previous clauses, wherein the one or more instructions further cause the processor to continuously or periodically obtain the benchmark biometric data and the in-vehicle biometric data.

The vehicle biometric system of any of the previous clauses, wherein the one or more vehicle action events include reducing a speed of a vehicle, initiate communication with an entity via a generated alert, controlling the vehicle to drive to a side of a road, or any combination thereof.

The vehicle biometric system of any of the previous clauses, wherein the one or more instructions further cause the processor to ascertain at least one of the occupant condition and the emotional state of a plurality of occupants.

A method, comprising: obtaining benchmark biometric data from a first data source; obtaining in-vehicle biometric data from a second data source; transmitting the benchmark biometric data from the first data source and the in-vehicle biometric data from the second source to a neural network; updating a model based on training data by processing the benchmark biometric data and the in-vehicle biometric data; transmitting the updated model; ascertaining at least one of an occupant condition and an emotional state of an occupant based on the updated model; and controlling one or more vehicle action events based on ascertaining at least one of the occupant condition and the emotional state of the occupant.

The method of the previous clause, further comprising pre-processing the benchmark biometric data and the in-vehicle biometric data to filter noise.

The method of any of the previous clauses, wherein the benchmark biometric data or the in-vehicle biometric data includes heart data, respiration data, eye information data, electrodermal activity data, or any combination thereof.

The method of any of the previous clauses, further comprising generating one or more prompts related to a state of the occupant; transmitting the one or more prompts related to the state of the occupant; and receiving, in response to the one or more prompts, one or more responses via user input.

The method of any of the previous clauses, wherein the user input includes audio input, textual input, gesture input, or any combination thereof.

The method of any of the previous clauses, further comprising updating the model based on the one or more responses via the user input.

The method of any of the previous clauses, further comprising continuously or periodically obtaining the benchmark biometric data and the in-vehicle biometric data.

The method of any of the previous clauses, wherein the one or more vehicle action events include reducing a speed of a vehicle, initiate communication with an entity via a generated alert, controlling the vehicle to drive to a side of a road, or any combination thereof.

The method of any of the previous clauses, further comprising ascertaining at least one of the occupant condition and the emotional state of a plurality of occupants.

A non-transitory computer-readable medium comprising instructions that, when executed by at least one processor, cause the at least one processor to perform one or more operations comprising: obtaining benchmark biometric data from a first data source; obtaining in-vehicle biometric data from a second data source; transmitting the benchmark biometric data from the first data source and the in-vehicle biometric data from the second source to a neural network; updating a model based on training data by processing the benchmark biometric data and the in-vehicle biometric data; transmitting the updated model; ascertaining at least one of an occupant condition and an emotional state of an occupant based on the updated model; and controlling one or more vehicle action events based on ascertaining at least one of the occupant condition and the emotional state of the occupant.

The non-transitory computer-readable medium of the previous clause, wherein the one or more vehicle action events include reducing a speed of a vehicle, initiate communication with an entity via a generated alert, controlling the vehicle to drive to a side of a road, or any combination thereof.

The preceding description is provided to enable any person skilled in the art to practice the various embodiments described herein. The examples discussed herein are not limiting of the scope, applicability, or embodiments set forth in the claims. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. For instance, the methods described may be performed in an order different from that described, and various steps may be added, omitted, or combined. Also, features described with respect to some aspects may be combined in some other aspects. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, the word “exemplary” means “serving as an example, instance, or illustration.” Any aspect described herein as “exemplary” is not necessarily to be construed as preferred or advantageous over other aspects.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c). Reference to an element in the singular is not intended to mean only one unless specifically so stated, but rather “one or more.” For example, reference to an element (e.g., “a processor,” “a memory,” etc.), unless otherwise specifically stated, should be understood to refer to one or more elements (e.g., “one or more processors,” “one or more memories,” etc.). The terms “set” and “group” are intended to include one or more elements, and may be used interchangeably with “one or more.” Where reference is made to one or more elements performing functions (e.g., steps of a method), one element may perform all functions, or more than one element may collectively perform the functions. When more than one element collectively performs the functions, each function need not be performed by each of those elements (e.g., different functions may be performed by different elements) and/or each function need not be performed in whole by only one element (e.g., different elements may perform different sub-functions of a function). Similarly, where reference is made to one or more elements configured to cause another element (e.g., an apparatus) to perform functions, one element may be configured to cause the other element to perform all functions, or more than one element may collectively be configured to cause the other element to perform the functions. Unless specifically stated otherwise, the term “some”refers to one or more.

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein include one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. A vehicle biometric system, comprising:
a processor; and
a non-transitory, processor-readable storage medium communicatively coupled to the processor, the non-transitory, processor-readable storage medium comprising one or more instructions stored thereon that, when executed, cause the processor to:
obtain benchmark biometric data from a first data source;
obtain in-vehicle biometric data from a second data source;
transmit the benchmark biometric data from the first data source and the in-vehicle biometric data from the second source to a neural network;
update a model based on training data by processing the benchmark biometric data and the in-vehicle biometric data;
transmit the updated model;
ascertain at least one of an occupant condition and an emotional state of an occupant based on the updated model; and
control one or more vehicle action events based on ascertaining at least one of the occupant condition and the emotional state of the occupant.

2. The vehicle biometric system of claim 1, wherein the one or more instructions further cause the processor to pre-process the benchmark biometric data and the in-vehicle biometric data to filter noise.

3. The vehicle biometric system of claim 1, wherein the benchmark biometric data or the in-vehicle biometric data includes heart data, respiration data, eye information data, electrodermal activity data, or any combination thereof.

4. The vehicle biometric system of claim 1, wherein the one or more instructions further cause the processor to:
generate one or more prompts related to a state of the occupant;
transmit the one or more prompts related to the state of the occupant; and
receive, in response to the one or more prompts, one or more responses via user input.

5. The vehicle biometric system of claim 4, wherein the user input includes audio input, textual input, gesture input, or any combination thereof.

6. The vehicle biometric system of claim 4, wherein the one or more instructions further cause the processor to update the model based on the one or more responses via the user input.

7. The vehicle biometric system of claim 1, wherein the one or more instructions further cause the processor to continuously or periodically obtain the benchmark biometric data and the in-vehicle biometric data.

8. The vehicle biometric system of claim 1, wherein the one or more vehicle action events include reducing a speed of a vehicle, initiate communication with an entity via a generated alert, controlling the vehicle to drive to a side of a road, or any combination thereof.

9. The vehicle biometric system of claim 1, wherein the one or more instructions further cause the processor to ascertain at least one of the occupant condition and the emotional state of a plurality of occupants.

10. A method, comprising:
obtaining benchmark biometric data from a first data source;
obtaining in-vehicle biometric data from a second data source;
transmitting the benchmark biometric data from the first data source and the in-vehicle biometric data from the second source to a neural network;
updating a model based on training data by processing the benchmark biometric data and the in-vehicle biometric data;
transmitting the updated model;
ascertaining at least one of an occupant condition and an emotional state of an occupant based on the updated model; and
controlling one or more vehicle action events based on ascertaining at least one of the occupant condition and the emotional state of the occupant.

11. The method of claim 10, further comprising pre-processing the benchmark biometric data and the in-vehicle biometric data to filter noise.

12. The method of claim 10, wherein the benchmark biometric data or the in-vehicle biometric data includes heart data, respiration data, eye information data, electrodermal activity data, or any combination thereof.

13. The method of claim 10, further comprising:
generating one or more prompts related to a state of the occupant;
transmitting the one or more prompts related to the state of the occupant; and
receiving, in response to the one or more prompts, one or more responses via user input.

14. The method of claim 13, wherein the user input includes audio input, textual input, gesture input, or any combination thereof.

15. The method of claim 13, further comprising updating the model based on the one or more responses via the user input.

16. The method of claim 10, further comprising continuously or periodically obtaining the benchmark biometric data and the in-vehicle biometric data.

17. The method of claim 10, wherein the one or more vehicle action events include reducing a speed of a vehicle, initiate communication with an entity via a generated alert, controlling the vehicle to drive to a side of a road, or any combination thereof.

18. The method of claim 10, further comprising ascertaining at least one of the occupant condition and the emotional state of a plurality of occupants.

19. A non-transitory computer-readable medium comprising instructions that, when executed by at least one processor, cause the at least one processor to perform one or more operations comprising:
obtaining benchmark biometric data from a first data source;
obtaining in-vehicle biometric data from a second data source;
transmitting the benchmark biometric data from the first data source and the in-vehicle biometric data from the second source to a neural network;
updating a model based on training data by processing the benchmark biometric data and the in-vehicle biometric data;
transmitting the updated model;
ascertaining at least one of an occupant condition and an emotional state of an occupant based on the updated model; and
controlling one or more vehicle action events based on ascertaining at least one of the occupant condition and the emotional state of the occupant.

20. The non-transitory computer-readable medium of claim 19, wherein the one or more vehicle action events include reducing a speed of a vehicle, initiate communication with an entity via a generated alert, controlling the vehicle to drive to a side of a road, or any combination thereof.
